(19) United States
(12) Patent Application Publication
(10) Pub. No.: US 2024/0091437 A1
(43) Pub. Date: Mar. 21, 2024

(54) DETECTION OF INFUSION SITE FAILURE

(71) Applicant: CareFusion 303, Inc., San Jose, CA (US)

(72) Inventors: Jason Andrew WINE, Brea, CA (US); Abin AUSTIN, Thrissur (IN); Kanjimpuredathil Muralikrishna MENON, Bangalore (IN); Narsi Reddy SANIKOMMU, Kanigiri (IN); Ankita JAWALE, Nashik (IN); Gene E. MASON, La Habra Heights, CA (US)

(73) Assignee: CareFusion 303, Inc., San Diego, CA (US)

(21) Appl. No.: 17/945,906

(22) Filed: Sep. 15, 2022

(51) Int. Cl. A61M 5/168

(52) U.S. Cl. CPC A61M 5/16836; A61M 2205/0294; A61M 2205/18; A61M 2205/3306; A61M 2205/3331; A61M 2230/04

(57) ABSTRACT

A first venous rhythm of a patient is detected above a venous insertion site when a venous catheter connected to an infusion set is inserted into the venous insertion site and, concurrently with the first venous rhythm, a second venous rhythm is detected at a location remote from the venous insertion site. An irregularity between the first and second venous rhythms is identified, and a failure of the venous catheter is determined based on the identified irregularity satisfying a irregularity threshold. An alarm may be provided on detecting the irregularity or the failure of the venous catheter.

### BACKGROUND

An incorrectly positioned catheter, or one that shifts to an incorrect position after placement in a vein, can lead to leakage of the infused fluid into surrounding tissues. Depending on the type of medication or fluid, the leakage may result in potentially serious complications, including harm to the patient. If the fluid is non-vesicant (e.g., does not typically irate tissue) then the leakage may be called an infiltration. If the fluid is a vesicant (e.g., a type of fluid that is known to irate tissue) then the leakage may be called an extravasation. Fluid leakage at or within an infusion site is not always immediately noticeable and may go undetected for extended periods of time.

### SUMMARY

According to various aspects of the subject technology, a method includes detecting a first venous rhythm of a patient above a venous insertion site of the patient when a venous catheter is inserted into the venous insertion site, the venous catheter being connected to an infusion set providing a fluid to the venous insertion site from a fluid source; detecting, concurrently with the first venous rhythm, a second venous rhythm at a location remote from the venous insertion site; identifying an irregularity between the first and second venous rhythms; and detecting a failure of the venous catheter based on the identified irregularity satisfying a irregularity threshold. Other aspects include corresponding systems, apparatus, and computer program products for implementation of the corresponding method and its features.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Description of Implementations below, in conjunction with the following drawings. Like reference numerals refer to corresponding parts throughout the figures and description.

FIG. 1 depicts an example infusion device, shown in use in its intended environment.

FIGS. 2A through 2D depict example infusion site failures detected by the subject technology.

FIG. 3 depicts an example pulsatile waveforms for detecting infusion site failures, according to various aspects of the subject technology.

FIG. 4 depicts an example processing flow diagram for detecting infusion site failures, according to various aspects of the subject technology.

FIG. 5 depicts a first example system for detecting infusion site failures, according to various aspects of the subject technology.

FIG. 6 depicts a second example system for detecting infusion site failures, according to various aspects of the subject technology.

FIG. 7 depicts a third example system for detecting infusion site failures, according to various aspects of the subject technology.

FIG. 8 depicts an example process for detecting infusion site failures, according to various aspects of the subject technology.

FIG. 9 is a conceptual diagram illustrating an example electronic system for detecting infusion site failures, according to various aspects of the subject technology.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

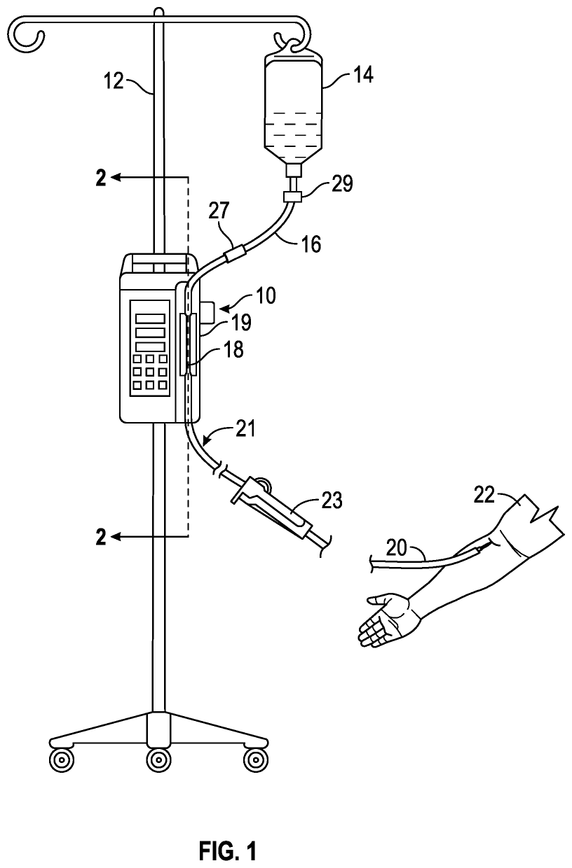

FIG. 1 depicts an example infusion device 10, shown in use in its intended environment, according to various aspects of the subject technology. In particular, the infusion device 10 is shown mounted to an intravenous (IV) pole 12 on which a fluid source 14 containing an IV fluid is held. The fluid source 14 is connected in fluid communication with an upstream fluid line 16. The fluid line 16 is a conventional IV infusion type tube typically used in a hospital or medical environment, and is made of any type of flexible tubing appropriate for use to infuse therapeutic fluids into a patient, such as polyvinylchloride (PVC). In some implementations, the flexible fluid line 18 is mounted in operative engagement with a peristaltic pumping apparatus 19, for propelling fluid through a downstream fluid line 20, for example, to a patient's arm 22.

It will be understood by those skilled in the art that the upstream fluid line 16, the flexible line 18, and the downstream fluid line 20 may be portions of a continuous length of flexible tubing, with the portions defined by the location of the peristaltic pump 19. For convenience, the continuous length of flexible tubing is indicated by numeral 21. A roller clamp 23 (e.g., configured to provide for mechanical compression of the line to block the flow) may be positioned on the downstream fluid line 20 between the pump 10 and the patient's arm 22. In this context, the term “upstream” refers to that portion of the flexible tubing that extends between the fluid source and peristaltic pump, and the term “downstream” refers to that portion of the flexible tubing that extends from the peristaltic pump to the patient.

Also shown in FIG. 1 is a secondary administration setup generally indicated by numeral 24. The secondary administration setup 24 includes a secondary fluid container 25 that may be filled with a second therapeutic fluid for infusion into the patient 22. Fluid from the secondary fluid container 25 flows through a secondary fluid line 26 into the fluid line 16 through a connector 27. A manually operated valve 28 is located in the secondary line 26 to control the flow of fluid flowing out of the secondary container 25 into the upstream fluid line 16. The one-way check valve 29 is disposed in the upstream fluid line 16 between the primary fluid container 14 and the connector 27, the one-way check valve is configured so that when the elevation of the fluid in the secondary container 25 is greater than that of the primary container, the differential pressure within line 16 closes the check valve and prevents secondary fluid from flowing into the primary container 14, and also prevents fluid from flowing out of primary container 14. Thus, the check valve 29 generally prevents mixing of the primary and secondary infusion fluids.

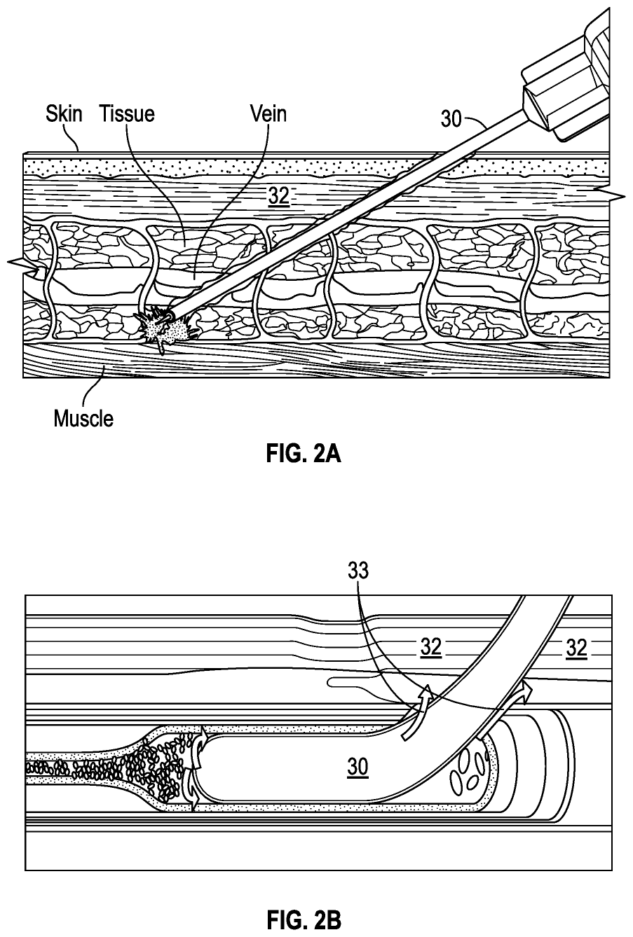

FIGS. 2A through 2D depict example infusion site failures detected by the subject technology. While the figures may depict the patient's arm, it should be understood that the subject technology may be used on other portions of the body. For example, the subject technology may be used to monitor an infusion site located on the torso of a patient. In FIG. 2A, the catheter 30 has been inserted through the epidermis 32 of a patient, entered a vein, and then punctured the wall of the vein (other than the entry puncture). In FIG. 2B, fluid from the vein is leaking from the venous insertion site 33 of the catheter 30.

Signs and symptoms may include fluid leakage, pain, redness, burning, pallor, no blood return, edema, decreased IV flow or flush, and the like. FIG. 2C depicts fluid 34 leaking from the infusion site, and redness 36 surrounding the site. FIG. 2D depicts redness and/or swelling 38, and clear topological damage 40, including blistering, to the area surrounding the infusion site.

Puncture and fluid leakage may be caused, for example, by vein fragility or clinician error, or from increased vein porosity. In some instances, the catheter 30 may back out of the insertion site, for example, when it is inserted or due to patient movement. Damage caused by infusion site failures can extend to involve nerves, tendons, and joints, and can continue for months after the initial injury. If treatment is delayed, surgical debridement, skin grafting, and even amputation may be the unfortunate consequences of the injury. The subject technology detects indications of fluid leakage, including extravasation and infiltration—leakage of an intravenously infused fluid, minimizing leakage of potentially damaging medications into the extravascular tissue around the site of infusion 22, thereby avoiding harm to the patient. In this regard, multiple sensors are used to monitor an infusion site and the signals compared to determine whether an infusion site failure (e.g., leakage) has occurred or is occurring.

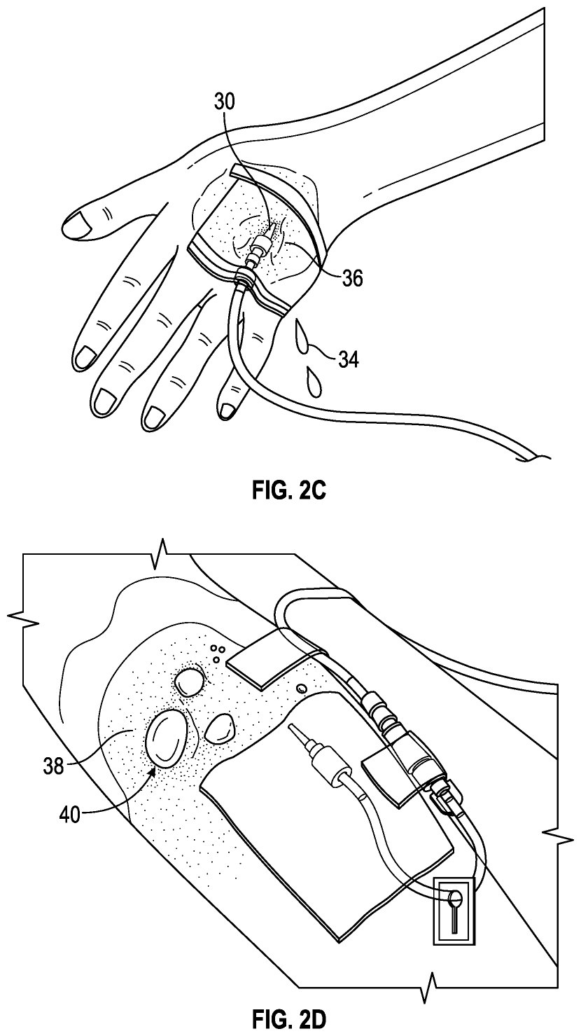

FIG. 3 depicts an example pulsatile waveforms for detecting infusion site failures, according to various aspects of the subject technology. As will be described further, the disclosed system detects a first venous rhythm of a patient above a venous insertion site of a catheter and a second venous rhythm at a location remote from the venous insertion site, and determines a failure of the catheter based on identifying an irregularity between the first and second venous rhythms. The depicted waveforms are indicative of these detected venous rhythms.

Peripheral venous waveforms can be captured by direct transduction through a peripheral catheter, non-invasive piezoelectric transduction, or gleaned from other waveforms such as the plethysmograph. Example raw venous waveforms in the time-domain are illustrated on the left, and in the frequency domain on the right. The top waveform is an example central venous waveform (CVP), followed by a peripheral venous waveform analysis (PIVA) based on signals acquired from direct transduction, and noninvasive waveform analysis (NIVA) with a piezo electric sensor at the bottom. f0 corresponds to the fundamental frequency which may be equal to the pulse rate. Higher harmonics of the pulse rate may be denoted by f1 and f2, as well as a low frequency component (frr) corresponding to the respiratory ra.

According to various implementation, pulse rates obtained based on different modalities (CVP, PIVA, and/or NIVA) may be compared to determine where there has been an infusion site failure. For example, a NIVA wave may be captured by a piezo electric sensor and compared to a peripheral venous waveform captured by direct transduction through a peripheral catheter. In the depicted examples, noninvasive waveforms may be captured by way of a piezo electric sensor(s) and/or photoplethysmography (PPG), thereby capturing and validating the peripheral venous pressure rhythm outside to detect infusion irregularities such as infiltration or extravasation. According to various implementations, the fundamental frequencies (f0) may be compared. However, in some implementations, a fundamental frequency (f0) may be compared to a higher harmonic (f1, f2, etc.).

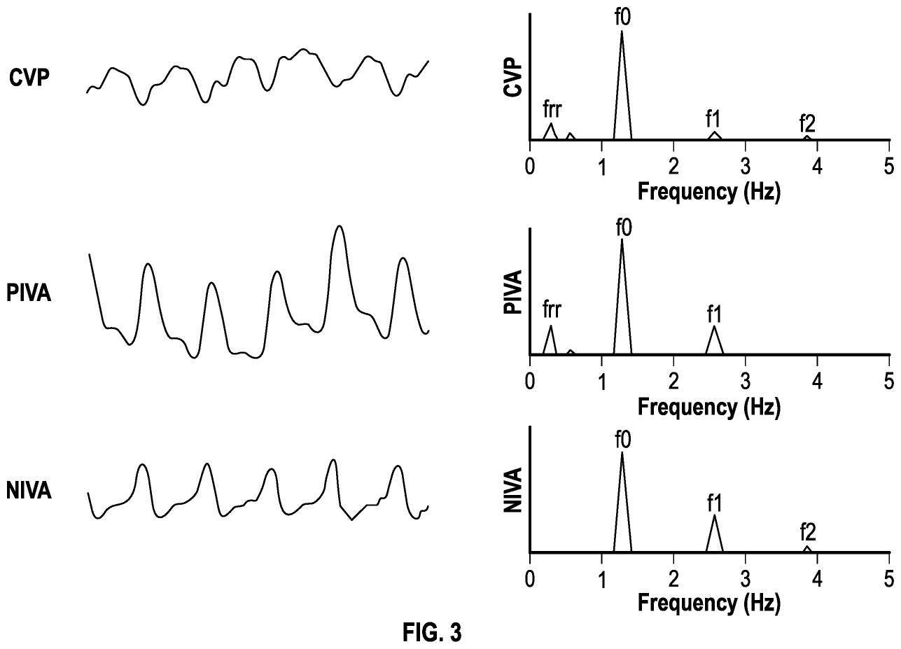

FIG. 4 depicts an example processing flow diagram for detecting infusion site failures, according to various aspects of the subject technology. According to various implementations, a first signal 402 indicative of a first venous rhythm is received by a first sensor 404, and a second signal 406 indicative of a second venous rhythm is received by a second sensor 408. As will be described further, the first and second sensors 404, 408 may each be a piezo electric sensor, a plethysmograph or photoplethysmograph such as an optical heart rate monitor, light sensor, or optical sensor for sensing light absorption. The first and second sensors 404, 408 receive the first and second signals 402, 406 concurrently, and pass them to a processor 410 for processing. The processor then processes the concurrent rhythmic signals and/or waveforms to identify an irregularity between the first and second venous rhythms.

The system may identify the irregularity by comparing the signals 402, 406. The signals (fundamental and/or higher harmonics) are expected to match under normal conditions. If the signals match (e.g., within an irregularity threshold) then no irregularity (at least initially) may be determined. If the two signals do not match (e.g., outside of an irregularity threshold) then an irregularity may be determined. For example, the first signal may be from a piezo electric sensor on the catheter, and the second signal may be from an optical heart rate monitor on the patient's wrist (e.g., on a smart watch). If the signal from the catheter does not match with the signal from the heart rate monitor then the system may detect an irregularity. Also, if one or more of the signals are not available, the system may determine a system fault and provide an alarm and/or perform diagnostic measures to determine whether the signal failure can be identified and corrected.

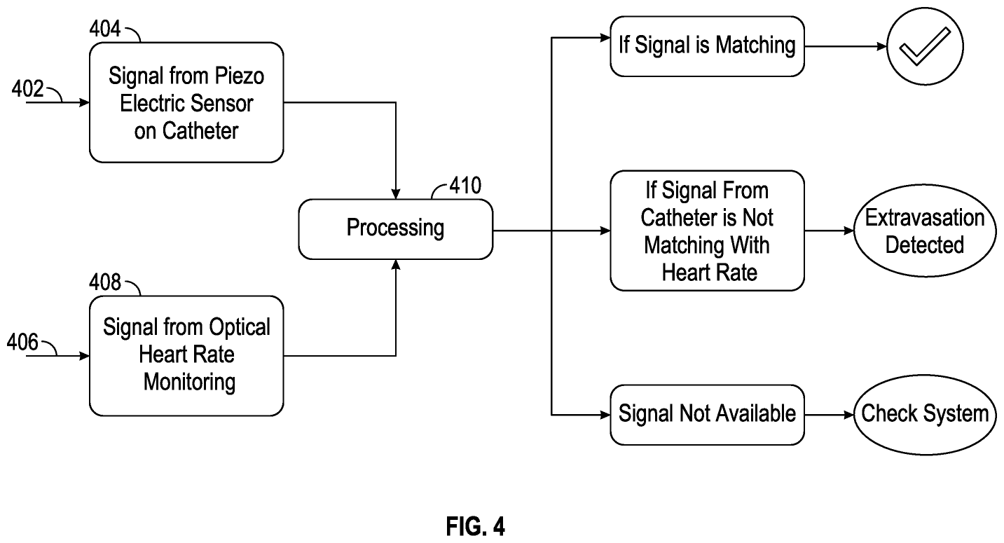

FIG. 5 depicts a first example system 500 for detecting infusion site failures, according to various aspects of the subject technology. An infusion set 502 is connected to a venous catheter, which may be inserted into a vein in the customary way by a clinician. The infusion set may then be connected to an downstream fluid line 20 (FIG. 1) by way of a luer or y-connector 503.

A pressure transducer (404, not shown) is provided in or attached to the venous catheter 504. In some implementations, the infusion set 502 is configured to position the transducer over the venous insertion site and at least a portion of the venous catheter 504 when the venous catheter 504 is inserted into the venous insertion site. For example, the infusion set 502 may be a winged infusion set or include a wing or stabilizer 506 for assigning insertion and stabilization of the catheter at a shallow angle. The wing or stabilizer 506 may include a noninvasive piezo electric sensor, or optical sensor, configured to monitor a pulsatile rhythm of the vein into which the catheter 504 is inserted.

In the depicted example, a peripheral venous pressure rhythm at the infusion site is detected by a pressure transducer 404 and compared to a second venous rhythm signal obtained by a photo plethysmograph 408. In some implementations, as will be described further, the catheter 504 may include a light emitting diode, and the first venous rhythm may be detected using a light detector in the wing or stabilizer 506. In the depicted example, the second venous rhythm is obtained from a optical heart rate monitor 408 in a smart watch 508. In some implementations, the second venous rhythm may be obtained using a pulse oximeter, for example attached to a fingertip (e.g., a sensor 408 within the pulse oximeter). The smart watch, pulse oximeter, or other secondary device responsible for detecting the second venous rhythm may be operably connected to the first sensor via a wire 510, which may be separated from or embedded within the infusion set 502.

In some implementations, the processor 410 may be implemented by the smart watch 508. In this regard, the system may include operating instructions stored on the watch 508 (e.g., in the form of an app) that receives data from the first and second sensors 404, 410, processes the data to compare the waveforms. If an irregularity or failure of the venous catheter is detected then an alert may be provided to the user. The alert may be in the form of an audio, visual, or haptic alarm. The alert may additionally or alternatively be provided to a connected computing device.

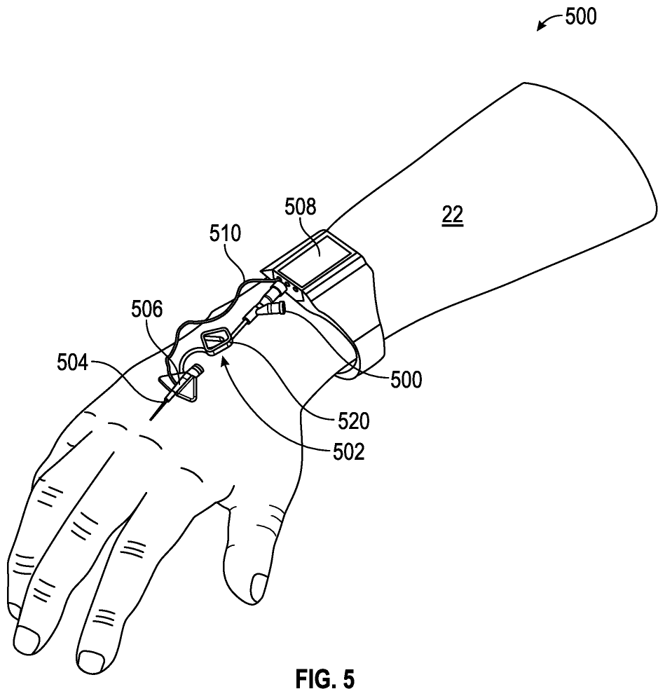

FIG. 6 depicts a second example system for detecting infusion site failures, according to various aspects of the subject technology. In some implementations, the infusion set of the disclosed infusion site failure detection system may include a housing 512 that rests on a surface of the skin 513 (or epidermis) above the infusion site. In this manner, the housing 512 may include one or more transducer(s) for detecting a venous rhythm associated with a vein 511 into which the catheter 504 is inserted. According to some implementations, the housing 512 may make up or be part of wing or stabilizer 506.

The example housing includes two light sensors 514, 516, and includes a power source 518 for an external pressure sensor 520. According to various implementations herein, sensor 514 and sensor 404 are interchangeable, while sensor 520 and sensor 408 are interchangeable. In the depicted example, a first venous rhythm of a patient is detected above the insertion site using a plethysmograph technique. For example, the housing 512 may include a photo plethysmograph to detect a pulse through the skin. With brief reference to FIG. 5, the pressure sensor 520 may be positioned on a portion of the fluid line of the infusion set 502.

According to various implementations, the first venous rhythm and second venous rhythm are compared to determine whether a threshold irregularity has been satisfied. For example, the two waveforms are compared to determine whether the waveforms match within a predetermined tolerance (e.g., greater than 70%, within one standard deviation on either side of the mean). In some implementations, a baseline amplitude of each signal may be determined for a predetermined period of time after the catheter is installed, for example, responsive to user input at a corresponding computing device indicating the catheter was inserted into the vein. If the baseline pulsatile amplitude of the second venous rhythm remains unchanged while a pulsatile amplitude of the first venous rhythm is declining then a leak may be indicated.

In some implementations, the second sensor 516 may be configured to sense changes in light absorption or color associated with an area of skin 521 adjacent to the infusion site (and the sensor 516). In some implementations, the second sensor 516 includes an ultrasound configured to sense a change in skin topography. When an irregularity is detected based on comparing sensors 514, 520, sensor 516 may be used to determine whether there has been a change in skin topography or light absorption adjacent to the venous insertion site. If no change is detected by sensor 516 then an infiltration may be determined. On the other hand, if a change is detected by sensor 516 then the system may determine that an extravasation has occurred. In some implementations, the system determines that an extravasation has occurred based on the change in topography or light absorption satisfying an extravasation threshold.

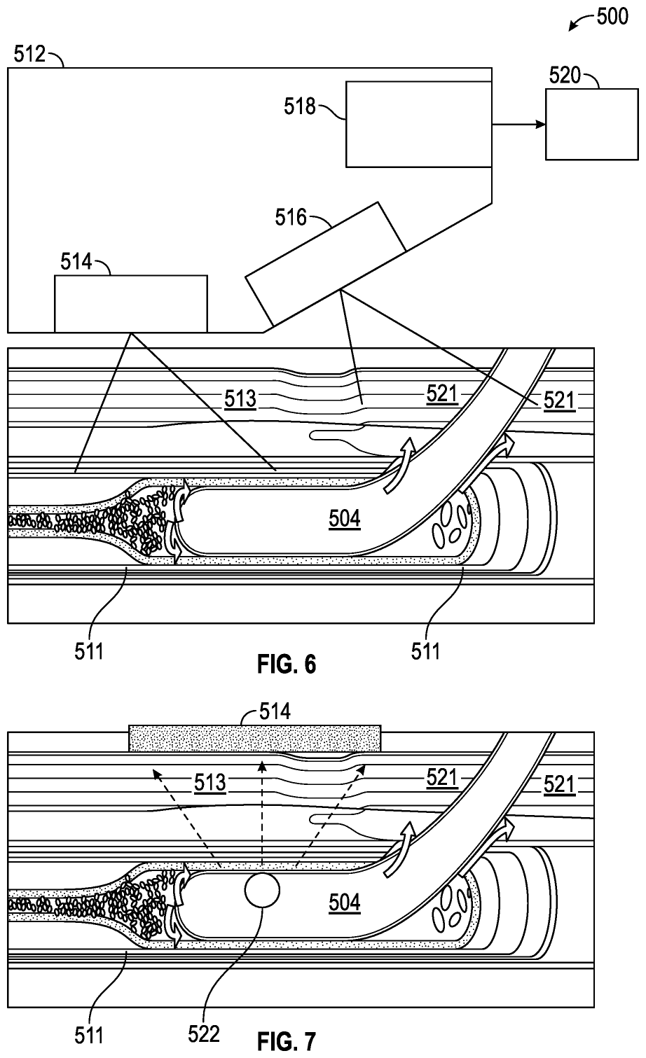

FIG. 7 depicts a third example system for detecting infusion site failures, according to various aspects of the subject technology. In the depicted example, sensor 514 includes a light detector configured to detect light emanating from under the epidermis. An emitter 522 (a light source) is embedded within a catheter 504, at a position such that when the catheter is inserted within a vein 511. According to some implementations, housing 512 (e.g., wing or stabilizer 506) may be connected to catheter 504 (see FIG. 5) such that the sensor 514 and light source 522 are aligned and/or such that light source 522 can be detected to a predetermined certainty by sensor 514.

In some implementations, the housing may include a processor 410 to process the data from the first and second sensors 404, 410. If an irregularity or failure of the venous catheter is detected then an alert may be provided to the user. The alert may be in the form of an audio, visual, or haptic alarm. The alert may additionally or alternatively be provided to a connected computing device.

According to various implementations, the light source 522 produces a constant amount of light, which can be baselined and established a default or baselined amount of light. If the catheter 504 is moved deeper into the subcutaneous tissue then the light absorbed by sensor 514 will change (e.g., decrease). For example, the pulsatile amplitude is represented by the amount of light reaching sensor 514, and a venous rhythm is detected based on detecting a time varying amplitude of the light source 522. If the amount of pulsating light decreases with respect to a baseline pulsatile amplitude then it may be determined that the catheter 504 has moved (to a side or deeper into the tissue). This may be particularly the case where the amplitude of the first venous rhythm declines with respect to a baseline pulsatile amplitude of the second venous rhythm (e.g., measured by sensor 520/408).

The system measures a pulse sinusoid based on light fluctuations, and may baseline the intensity of light detected at the sensor 514 based on the average of the pulse sinusoid. If the intensity of the light source 522 increases (as measured by the sensor 514) then the system may determine that the venous catheter 504 has moved closer to the surface of the skin. If the intensity of the light (as measured by the sensor 514) decreases then the system may determine that the venous catheter 504 has moved or punctured deeper into the tissue. Determination of whether the catheter has moved closer or away from the surface may be based on determining whether the increase or decrease in light satisfies a threshold for the increase or decrease, respectively.

If the tip of the catheter 504 clogs or a blood clot forms in the vein 511 then the pressure amplitude or amplitude of the light source 522 may decrease over time. In some implementations, when such a decline is detected (e.g., when it passes the threshold for a failure of the catheter), the amplitude of the signal may be checked against a baseline signal. For example, the system may baseline the optical signal; i.e., determine the baseline light intensity (e.g., irrespective of modulation due to a pulse), or average intensity over a period of time. When checked, if the baseline remains unchanged while the pulsatile amplitude is declining then the system may determine that the change in amplitude is from an occlusion rather than from movement of the catheter 504. If the baseline amount of light is also changing then the system may determine that the catheter has moved. The average of the pulse sinusoid is not expected to change while the catheter is in the vein, even if the amplitude declines due to clogging.

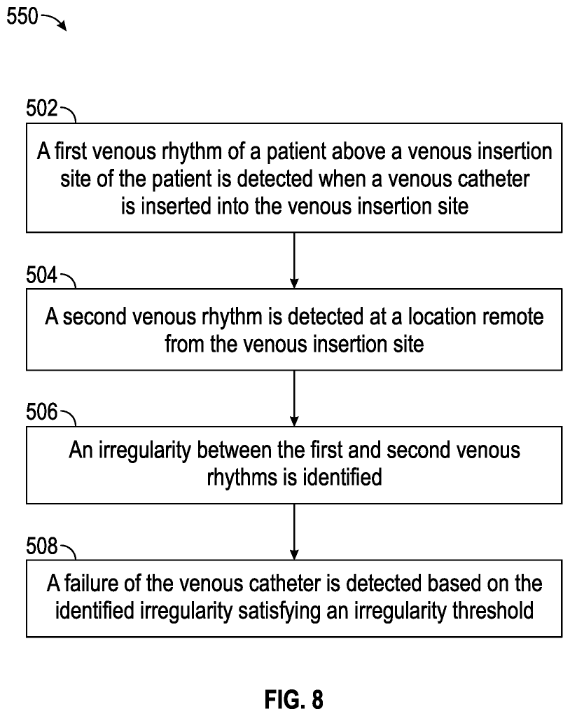

FIG. 8 depicts an example process 550 for detecting an infusion site failure, according to aspects of the subject technology. For explanatory purposes, the various blocks of example process 550 are described herein with reference to FIGS. 1-7, and the components and/or processes described herein. The one or more of the blocks of process 550 may be implemented, for example, by one or more computing devices including, for example, in housing 512 or smart watch 508 or an operably connected computing device (e.g., wirelessly connected to sensors 404, 408). In some implementations, one or more of the blocks may be implemented based on one or more algorithms. In some implementations, one or more of the blocks may be implemented apart from other blocks, and by one or more different processors or devices. Further for explanatory purposes, the blocks of example process 550 are described as occurring in serial, or linearly. However, multiple blocks of example process 550 may occur in parallel. In addition, the blocks of example process 550 need not be performed in the order shown and/or one or more of the blocks of example process 550 need not be performed.

In the depicted example, a first venous rhythm of a patient is detected above a venous insertion site of the patient when a venous catheter is inserted into the venous insertion site (552). As described previously, the venous catheter is connected to an infusion set 502 providing a fluid to the venous insertion site from a fluid source. With reference to FIG. 1, the infusion set 502 configured to connect the venous catheter to an infusion line 20 providing an infusion fluid from a fluid source, such as an infusion device 10. The venous rhythm may be detected, for example, by a first sensor 404, 514 above or proximate the infusion site. In some implementations, the first sensor 404, 514 includes a transducer or a piezo electric circuit configured to detect a time varying pressure within the fluid passing through the infusion set 502.

In some implementations (e.g., in FIG. 6), the infusion set 502 is configured to position the transducer over the venous insertion site and at least a portion of the venous catheter 504 when the venous catheter 504 is inserted into the venous insertion site (e.g., within a vein). In some implementations, the transducer may be placed remote from the insertion site; for example, at a location 520 attached to the infusion set 502 a couple inches away from the venous insertion site, as shown in FIG. 5.

As described previously, in some implementations, the transducer 514 includes a light detector configured to detect a light source 522 that emits a light towards the surface of the skin and towards the transducer 514. In this manner, a time varying change in the light source through subcutaneous tissue between the light source and the transducer. Accordingly, the peaks of the detected sinusoid (e.g., light, pressure, or other modality) may be representative of the first venous rhythm.

Concurrently with the first venous rhythm, a second venous rhythm is detected at a location remote from the venous insertion site (554). In some implementations, the second venous rhythm is detected using a second sensor 408 such as a photoplethysmograph. For example, a heart rate monitor of a smart watch may be used as the second sensor. The second sensor being remote from the venous insertion site (and/or the first sensor) includes the two sensors being a couple inches between the second sensor and the venous insertion site (and/or the first sensor).

An irregularity between the first and second venous rhythms is identified (556), and a failure of the venous catheter detected based on the identified irregularity satisfying a irregularity threshold (558). The irregularity may include, for example, a difference in frequency between the first and second venous rhythms. In some implementations, the irregularity may be a difference in amplitude in the first venous rhythm from a baseline amplitude of the first or second venous rhythm. For example, the pulsatile amplitude of the first venous rhythm be identified as declining with respect to a baseline pulsatile amplitude of the second venous rhythm. In some implementations, waveforms for each of the venous rhythms may be generate or determined and an intelligent algorithm may utilize pattern matching to compare the waveforms.

According to various implementations, the failure of the venous catheter may include a fluid leakage from the infusion site, or into tissue surrounding the infusion site. In one example, a fluid leak is detected when the pulsatile amplitude of the first venous rhythm declines with respect to a baseline pulsatile amplitude of the second venous rhythm by a threshold decline (e.g., more than 25%).

In some implementations, the first venous rhythm is detected based on detecting a time varying amplitude of a light source 522. Based on an intensity of the light source increasing to satisfy a first threshold, the system may determine that the venous catheter has moved, is moving, closer to a surface of the skin. Based on the intensity of the light source decreasing to satisfy a second threshold, the system may determine that the venous catheter has punctured deeper into the tissue beneath the skin.

In some implementations, the system may make a determination as to whether a declining pulsatile amplitude of the first venous rhythm is due to a fluid leak, such as an infiltration or extravasation, or due to an occlusion within the vein 511. An occlusion may be detected associated based on determining that a baseline pulsatile amplitude of the first venous rhythm remains unchanged while the pulsatile amplitude of the first venous rhythm is declining.

In some implementations, the system may make a determination as to whether the declining pulsatile amplitude of the first venous rhythm is due to a fluid infiltration or an extravasation by activation of a second sensor 516 to detect whether there has been a change in topography, color, or light absorption of a portion of the epidermis 521 adjacent to the venous insertion site. In this regard, the second sensor 516 may include a light sensor configured to detect color or light absorption, or an ultrasound configured to measure a topography of the epidermis 521. The sensor 516 may be initially activated for a predetermined period of time at the start of the infusion (e.g., when receiving user input indicating the infusion has started or the catheter was inserted) to baseline the characteristics (light, topography, color) of the surface of the epidermis. On detecting an irregularity between the first sensor 404, 514 and second sensor 408, 520 (e.g., the two venous rhythms or waveforms do not match), the sensor 516 may be read to determine whether the current characteristics match prior (stored) characteristics. In some implementations, the system (e.g., the algorithm) determines whether the current and prior characteristics match within a predetermined threshold tolerance. Accordingly, when the pulsatile amplitude of the first venous rhythm is declining while the pulsatile amplitude of the second venous rhythm remains relatively unchanged, the system may determine an extravasation has occurred.

Many of the above-described example process 550, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

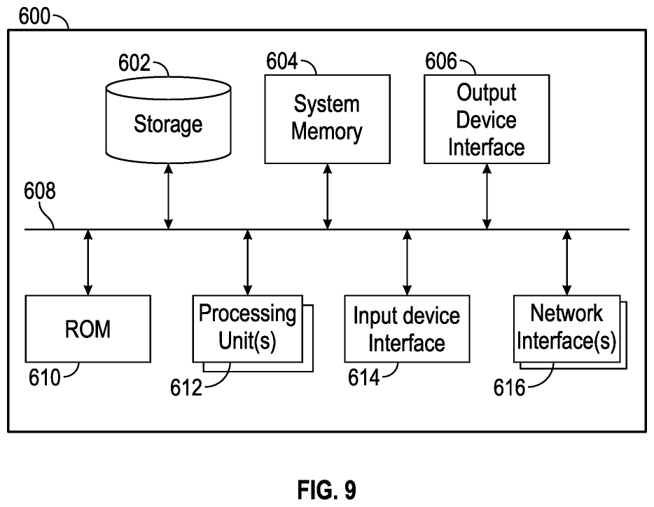

FIG. 9 is a conceptual diagram illustrating an example electronic system 600 for detecting an infusion site failure, according to aspects of the subject technology. Electronic system 600 may be a computing device for execution of software associated with one or more portions or steps of method 500, or components and methods provided by FIGS. 1-8, including but not limited to computing hardware within infusion device 10, or smart watch 508, housing 512, and/or any computing devices or associated terminals disclosed herein.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and methods previously described.

Bus 608 collectively represents all system, peripheral, and chipset buses that communicatively connect the numerous internal devices of electronic system 600. For instance, bus 408 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or WiFi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software, depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identification.

Clause 1. A method for detecting an infusion site failure, comprising: detecting a first venous rhythm of a patient above a venous insertion site of the patient when a venous catheter is inserted into the venous insertion site, the venous catheter being connected to an infusion set providing a fluid to the venous insertion site from a fluid source; detecting, concurrently with the first venous rhythm, a second venous rhythm at a location remote from the venous insertion site; identifying an irregularity between the first and second venous rhythms; and detecting a failure of the venous catheter based on the identified irregularity satisfying a irregularity threshold.

Clause 2. The method of Clause 1, wherein identifying the irregularity comprises: detecting a leakage of a fluid infused into tissue proximate the venous insertion site based on a pulsatile amplitude of the first venous rhythm declining with respect to a baseline pulsatile amplitude.

Clause 3. The method of Clause 2, further comprising: wherein the first venous rhythm is detected based on detecting a time varying amplitude of a light source; determining that the venous catheter has moved closer to a surface of the skin based on an intensity of the light source increasing to satisfy a first threshold; determining that the venous catheter has punctured deeper into the tissue based on the intensity of the light source decreasing to satisfy a second threshold.

Clause 4. The method of any one of Clauses 1 through 3, further comprising: detecting an occlusion associated with the venous insertion site based on a baseline light intensity of the first venous rhythm remaining unchanged while a pulsatile amplitude of the first venous rhythm is declining.

Clause 5. The method of any one of Clauses 1 through 4, further comprising: detecting, when the irregularity is identified, a change in topography or light absorption of a portion of epidermis adjacent the venous insertion site; determining that an extravasation has occurred based on the change in topography or light absorption satisfying an extravasation threshold.

Clause 6. The method of any one of Clauses 1 through 5, further comprising: providing audio, visual, or haptic alarm responsive to detecting the failure of the venous catheter.

Clause 7. The method of any one of Clauses 1 through 6, further comprising: providing an infusion set configured to connect the venous catheter to an infusion line providing an infusion fluid from a fluid source, the infusion set comprising a transducer configured to detect the first venous rhythm.

Clause 8. The method of Clause 7, wherein the transducer comprises a piezo electric circuit, the method further comprising: detecting the first venous rhythm by using the piezo electric circuit to detect a time varying pressure within the fluid passing through the infusion set.

Clause 9. The method of Clause 7, wherein the infusion set is configured to position the transducer over the venous insertion site and at least a portion of the venous catheter when the venous catheter is inserted into the venous insertion site, wherein the venous catheter comprises a light source, and the transducer comprises a light detector, the method further comprising: detecting the first venous rhythm by using the light detector to detect a time varying change in the light source through subcutaneous tissue between the light source and the transducer.

Clause 10. The method of any one of Clauses 1 through 9, further comprising: detecting the second venous rhythm using photoplethysmography.

Clause 11. A system for detecting an infusion site failure, comprising: one or more processors; and a memory device having computer-readable instructions stored thereon that, when executed by the one or more processors, perform a method according to any one of Clauses 1 through 7.

Clause 12. The system of Clause 11, further comprising: a pressure transducer; and a photoplethysmograph, wherein one of the first and second venous rhythms is detected by the pressure transducer and one of the first and second venous rhythms is detected by the photoplethysmograph.

Clause 13. The system of Clause 11, further comprising: a piezo electric transducer; and a photoplethysmograph, wherein one of the first and second venous rhythms is detected by the piezo electric transducer and one of the first and second venous rhythms is detected by the photoplethysmograph.

Clause 14. The system of Clause 11, further comprising: a light sensor; and a photoplethysmograph, wherein the first venous rhythm is detected by the light sensor based on detecting a time varying amplitude of a light source connected to or inside a catheter, and the second venous rhythm is detected by the photoplethysmograph.

Clause 15. A non-transitory computer-readable medium storing instructions thereon that, when executed by one or more processors, perform a method according to any one of Clauses 1 through 10.

Further Consideration:

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented. The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML, document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A method for detecting an infusion site failure, comprising:
detecting a first venous rhythm of a patient above a venous insertion site of the patient when a venous catheter is inserted into the venous insertion site, the venous catheter being connected to an infusion set providing a fluid to the venous insertion site from a fluid source;
detecting, concurrently with the first venous rhythm, a second venous rhythm at a location remote from the venous insertion site;
identifying an irregularity between the first and second venous rhythms; and
detecting a failure of the venous catheter based on the identified irregularity satisfying a irregularity threshold.

2. The method of claim 1, wherein identifying the irregularity comprises:
detecting a leakage of a fluid infused into tissue proximate the venous insertion site based on a pulsatile amplitude of the first venous rhythm declining with respect to a baseline pulsatile amplitude.

3. The method of claim 2, further comprising:
wherein the first venous rhythm is detected based on detecting a time varying amplitude of a light source;
determining that the venous catheter has moved closer to a surface of the skin based on an intensity of the light source increasing to satisfy a first threshold;
determining that the venous catheter has punctured deeper into the tissue based on the intensity of the light source decreasing to satisfy a second threshold.

4. The method of claim 1, further comprising:
detecting an occlusion associated with the venous insertion site based on a baseline light intensity of the first venous rhythm remaining unchanged while a pulsatile amplitude of the first venous rhythm is declining.

5. The method of claim 1, further comprising:
providing an infusion set configured to connect the venous catheter to an infusion line providing an infusion fluid from a fluid source, the infusion set comprising a transducer configured to detect the first venous rhythm.

6. The method of claim 5, wherein the transducer comprises a piezo electric circuit, the method further comprising:
detecting the first venous rhythm by using the piezo electric circuit to detect a time varying pressure within the fluid passing through the infusion set.

7. The method of claim 6, further comprising:
detecting the second venous rhythm using photoplethysmography.

8. The method of claim 5, wherein the infusion set is configured to position the transducer over the venous insertion site and at least a portion of the venous catheter when the venous catheter is inserted into the venous insertion site,
wherein the venous catheter comprises a light source, and the transducer comprises a light detector, the method further comprising:
detecting the first venous rhythm by using the light detector to detect a time varying change in the light source through subcutaneous tissue between the light source and the transducer.

9. The method of claim 8, further comprising:
detecting the second venous rhythm using photoplethysmography.

10. The method of claim 1, further comprising:
detecting, when the irregularity is identified, a change in topography or light absorption of a portion of epidermis adjacent the venous insertion site;
determining that an extravasation has occurred based on the change in topography or light absorption satisfying an extravasation threshold.

11. The method of claim 1, further comprising:
providing audio, visual, or haptic alarm responsive to detecting the failure of the venous catheter.

12. A system for detecting an infusion site failure, comprising:
one or more processors; and
a memory device having computer-readable instructions stored thereon that, when executed by the one or more processors, perform a method according to claim 1.

13. The system of claim 12, further comprising:
a pressure transducer; and
a photoplethysmograph,
wherein one of the first and second venous rhythms is detected by the pressure transducer and one of the first and second venous rhythms is detected by the photoplethysmograph.

14. The system of claim 12, further comprising:
a piezo electric transducer; and
a photoplethysmograph,
wherein one of the first and second venous rhythms is detected by the piezo electric transducer and one of the first and second venous rhythms is detected by the photoplethysmograph.

15. The system of claim 12, further comprising:
a light sensor; and
a photoplethysmograph,
wherein the first venous rhythm is detected by the light sensor based on detecting a time varying amplitude of a light source connected to or inside a catheter, and the second venous rhythm is detected by the photoplethysmograph.

16. A non-transitory computer-readable medium storing instructions thereon that, when executed by one or more processors, perform a method according to claim 1.
